(19) United States
(12) Patent Application Publication
(10) Pub. No.: US 2024/0415695 A1
(43) Pub. Date: Dec. 19, 2024

(54) DEVICE FOR COLD COMPRESS AND HOT COMPRESS

(71) Applicant: Xiamen Ubest Electronic Technology Co., Ltd., Xiamen (CN)

(72) Inventors: Yuci CHEN, Xiamen (CN); Kunlin CHEN, Xiamen (CN)

(73) Assignee: Xiamen Ubest Electronic Technology Co., Ltd., Xiamen (CN)

(21) Appl. No.: 18/446,757

(22) Filed: Aug. 9, 2023

(30) Foreign Application Priority Data
Jun. 14, 2023 (CN) 202321512812.5

(51) Int. Cl. A61F 7/00

(52) U.S. Cl. CPC A61F 7/0085

(57) ABSTRACT

A device for cold compress and hot compress includes a compress bag, a control device, and a heat dissipation device. The compress bag includes a semiconductor chilling plate and a working surface, and the semiconductor chilling plate is disposed on the working surface; and the heat dissipation device includes a cooling medium circulation device, the cooling medium circulation device includes a circulation passage and a cooling medium, and the circulation passage is formed in the compress bag and is adjacent to one side, away from the working surface, of the semiconductor chilling plate. Through rapid cooling and heating of the semiconductor chilling plate, temperature adjustment on the working surface of the compress bag is achieved to meet the demand for cold compress and hot compress of a user; the semiconductor chilling plate is directly disposed on the working surface of the compress bag.

### CROSS-REFERENCE TO RELATED APPLICATIONS

This application claims priority to Chinese Patent Application No. 202321512812.5, filed on Jun. 14, 2023, the content of which is incorporated herein by reference in its entirety.

### TECHNICAL FIELD

The present application relates to the field of medical care equipment, and particularly relates to a device for cold compress and hot compress.

### BACKGROUND

A device for cold compress and hot compress can be used for heating and cooling parts of a human body. By replacing conventional temperature adjustment means such as towel cold compress or hot compress, and ice water or warm water soaking with cold and hot adjustment of the device itself, it is greatly convenient for a user to use the device under different scene requirements. In Chinese application patent with the application number of CN202123352242.1, a sub-hypothermia pneumatic massage device for recovering after exercise is disclosed, and hot compress is achieved by heating a wearing part by means of electrification and heating of a carbon fiber heating plate; cold compress is achieved by filling the wearing part with ice water through a water cooling device; however, the device has a cumbersome cold-hot switching process, and cannot respond quickly, especially for switching from hot compress to cold compress, a large amount of cooling water needs to be cooled in advance and then is transferred to the wearing part through an external pipeline, and the cooling water is likely to exchange heat with the outside in the part of the external pipeline, thereby weakening the heat exchange efficiency at the wearing part and resulting in a big energy loss.

### SUMMARY

The technical problem to be solved by the present application is to provide a device for cold compress and hot compress which can achieve rapid cooling and heating adjustment, and is high in heat exchange efficiency.

In order to solve the technical problem above, the technical solution adopted by the present application is as follows: a device for cold compress and hot compress includes a compress bag, a control device, and a heat dissipation device, wherein the compress bag includes a semiconductor chilling plate and a working surface for being attached to a human body, and the semiconductor chilling plate is disposed on the working surface; the heat dissipation device includes a cooling medium circulation device, the cooling medium circulation device includes a circulation passage and a cooling medium in the circulation passage, and the circulation passage is formed in the compress bag and is adjacent to one side, away from the working surface, of the semiconductor chilling plate; and the control device is electrically connected to the semiconductor chilling plate and the heat dissipation device.

Further, the compress bag further includes a circuit substrate disposed corresponding to the semiconductor chilling plate, the semiconductor chilling plate is disposed on the circuit substrate, and the circuit substrate is electrically connected to the control device.

Further, the cooling medium circulation device further includes a storage tank, a pumping device, and an external connecting pipe, and the external connecting pipe is connected to the circulation passage in the compress bag and the storage tank; and the pumping device is disposed on one side of the storage tank and electrically connected to the control device.

Further, the heat dissipation device further includes a secondary heat dissipation device, and the cooling medium flows through the secondary heat dissipation device; and the secondary heat dissipation device is electrically connected to the control device.

Further, the secondary heat dissipation device includes a radiator and a heat dissipation fan, the cooling medium flows through the radiator, and the radiator is disposed on an air outlet path of the heat dissipation fan.

Further, the device for cold compress and hot compress further includes a strap, wherein the strap is removably connected to the compress bag.

The present application has the following beneficial effects: through rapid cooling and heating of the semiconductor chilling plate, temperature adjustment on the working surface of the compress bag is achieved to meet the demand for cold compress and hot compress of a user; the semiconductor chilling plate is directly disposed on the working surface of the compress bag, so as to ensure that temperature adjustment on the semiconductor chilling plate can directly act on a desired part, and improve the heat exchange efficiency; and by disposing the cooling medium circulation device, working heat of the adjacent semiconductor chilling plate during cooling is taken away by flowing of the cooling medium in the circulation passage in the compress bag, so as to rapidly dissipate heat of the whole device to ensure that a working temperature is normal; the cooling medium can exchange heat with heat at a cold end on the other side of the semiconductor chilling plate during heating of the semiconductor chilling plate, so as to ensure temperature balance of the whole device, and prevent temperatures at two ends of the semiconductor chilling plate from affecting each other; and the cooling medium can be cooling water, and the cooling water in a fluid form can satisfy efficient heat exchange in various models after the compress bag is attached to the human body.

### BRIEF DESCRIPTION OF THE DRAWINGS

FIG. 1 is a schematic structural diagram of an interior of a compress bag of a device for cold compress and hot compress according to an embodiment of the present application.

FIG. 2 is a schematic structural diagram of a housing of the device for cold compress and hot compress according to an embodiment of the present application.

FIG. 3 is a schematic structural diagram of an interior of the housing of the device for cold compress and hot compress according to an embodiment of the present application.

FIG. 4 is a schematic diagram of a flowing direction of a cooling medium of the device for cold compress and hot compress according to an embodiment of the present application.

FIG. 5 is a schematic structural diagram of the device for cold compress and hot compress with the compress bag and a strap being used for shoulder care according to an embodiment of the present application.

FIG. 6 is a schematic structural diagram of the device for cold compress and hot compress with the compress bag and the strap being used for waist care according to an embodiment of the present application.

FIG. 7 is a schematic structural diagram of the device for cold compress and hot compress with the compress bag and the strap being used for leg care according to an embodiment of the present application.

### DETAILED DESCRIPTION OF THE EMBODIMENTS

In order to explain the technical contents, achieved objects and effects of the present application in detail, the following description is made in conjunction with the embodiments and accompanying drawings.

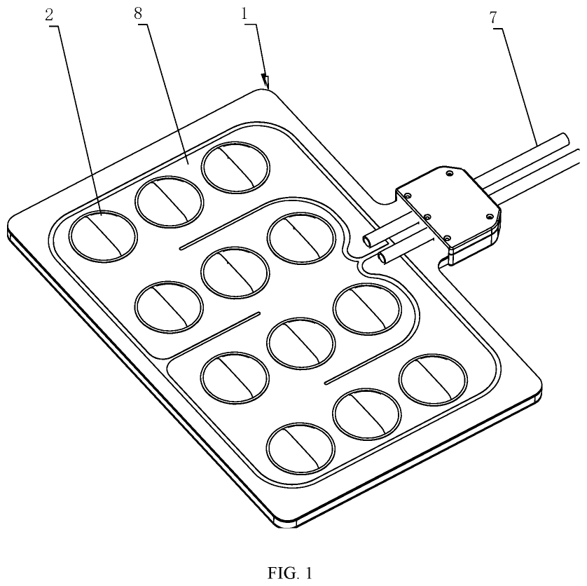

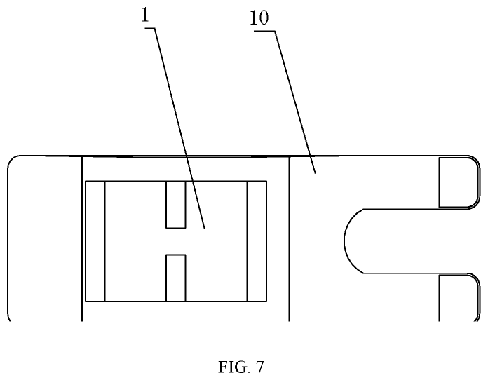

With reference to FIG. 1 to FIG. 7, a device for cold compress and hot compress includes a compress bag 1, a control device 3, and a heat dissipation device, wherein the compress bag 1 includes a semiconductor chilling plate 2 and a working surface for being attached to a human body, and the semiconductor chilling plate 2 is disposed on the working surface; the heat dissipation device includes a cooling medium circulation device, the cooling medium circulation device includes a circulation passage 4 and a cooling medium in the circulation passage 4, and the circulation passage 4 is formed in the compress bag 1 and is adjacent to one side, away from the working surface, of the semiconductor chilling plate 2; and the control device 3 is electrically connected to the semiconductor chilling plate 2 and the heat dissipation device.

It can be seen from the description above that the beneficial effects of the present application are as follows: through rapid cooling and heating of the semiconductor chilling plate 2, temperature adjustment on the working surface of the compress bag 1 is achieved to meet the demand for cold compress and hot compress of a user; the semiconductor chilling plate 2 is directly disposed on the working surface of the compress bag 1, so as to ensure that temperature adjustment on the semiconductor chilling plate 2 can directly act on a desired part, and improve the heat exchange efficiency; and by disposing the cooling medium circulation device, working heat of the adjacent semiconductor chilling plate 2 during cooling is taken away by flowing of the cooling medium in the circulation passage 4 in the compress bag 1, so as to rapidly dissipate heat of the whole device to ensure that a working temperature is normal; while the cooling medium can exchange heat with heat at a cold end on the other side of the semiconductor chilling plate 2 during heating of the semiconductor chilling plate 2, so as to ensure temperature balance of the whole device, and prevent temperatures at two ends of the semiconductor chilling plate 2 from affecting each other; and the cooling medium can be cooling water, and the cooling water in a fluid form can satisfy efficient heat exchange in various models after the compress bag 1 is attached to the human body.

Further, the compress bag 1 further includes a circuit substrate 8 disposed corresponding to the semiconductor chilling plate 2, the semiconductor chilling plate 2 is disposed on the circuit substrate 8, and the circuit substrate 8 is electrically connected to the control device 3.

It can be seen from the description above that the circuit substrate 8 is electrically connected to the control device 3, so as to control the cooling and heating of the semiconductor chilling plate 2.

Further, the cooling medium circulation device further includes a storage tank 5, a pumping device 6, and an external connecting pipe 7, and the external connecting pipe 7 is connected to the circulation passage 4 in the compress bag 1 and the storage tank 5; and the pumping device 6 is disposed on one side of the storage tank 5 and electrically connected to the control device 3.

It can be seen from the description above that the storage tank 5 is used for storing the cooling medium; the semiconductor chilling plate 2 is controlled by the control device 3 during working, and the cooling medium in the storage tank 5 is pumped into the circulation passage 4 by the pumping device 6 through the external connecting pipe 7 to rapidly take away the working heat of the semiconductor chilling plate 2.

Further, the heat dissipation device further includes a secondary heat dissipation device, and the cooling medium flows through the secondary heat dissipation device; and the secondary heat dissipation device is electrically connected to the control device 3.

It can be seen from the description above that the cooling of the cooling medium is accelerated by disposing the secondary heat dissipation device, so as to ensure that the working temperature of the semiconductor chilling plate 2 is normal.

Further, the secondary heat dissipation device includes a radiator 11 and a heat dissipation fan 9, the cooling medium flows through the radiator 11, and the radiator 11 is disposed on an air outlet path of the heat dissipation fan 9.

It can be seen from the description above that after the cooling medium flows through the semiconductor chilling plate 2 and takes away the heat, the cooling medium flows into the radiator 11 for heat dissipation and cooling, and the heat dissipation fan 9 blows the radiator 11 for heat dissipation; wherein the radiator 11 and the heat dissipation fan 9 can be connected to parts such as the circulation passage 4, the external connecting pipe 7, or the storage tank 5, so that the cooling medium can be rapidly cooled for subsequent recycling.

Further, the device for cold compress and hot compress further includes a strap 10, wherein the strap 10 is removably connected to the compress bag 1.

It can be seen from the description above that the design of the removable connection of the strap 10 enables the compress bag 1 to be rapidly attached to a part of the human body requiring care fixedly.

With reference to FIG. 1 to FIG. 7, example 1 of the present application is as follows.

An application scenario of the present application is as follows: when a part of a human body requires cold compress or hot compress, conventional equipment with cold compress and hot compress functions has a cumbersome cold-hot switching process, and cannot respond quickly, especially for switching from hot compress to cold compress, a large amount of cooling water needs to be cooled in advance and then is transferred to the wearing part through an external pipeline, and the cooling water is likely to exchange heat with the outside in the part of the external pipeline, thereby weakening the heat exchange efficiency at the wearing part and resulting in a big energy loss.

As shown in FIG. 1 to FIG. 7, a device for cold compress and hot compress of this example includes a compress bag 1, a strap 10, a control device 3, a heat dissipation device, and a housing.

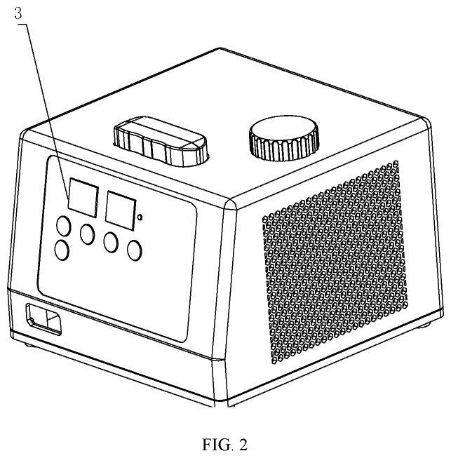

As shown in FIG. 2, the heat dissipation device includes a cooling medium circulation device and a secondary heat dissipation device, wherein the cooling medium circulation device includes a circulation passage 4, a cooling medium flowing in the circulation passage 4, a storage tank 5, a pumping device 6, and an external connecting pipe 7.

As shown in FIG. 1, specifically, the compress bag 1 consists of a plurality of the semiconductor chilling plate 2, circuit substrates 8, and removable cloth bags externally sleeved on the semiconductor chilling plate 2 and the circuit substrates 8, and the circulation passage 4 is formed in the compress bag 1.

Wherein working ends of the semiconductor chilling plates 2 are adjacent to a working surface, attached to a human body, on the compress bag 1; each of the circuit substrates 8 is provided with at least one semiconductor chilling plate 2, the semiconductor chilling plate is a common semiconductor element which can be used for cooling and heating on the market, and in this example, the type of the semiconductor chilling plate is TES1-4902;

the circuit substrate 8 is electrically connected to the control device 3 through an electric wire, the circuit substrate 8 is preferably an FPC, and cooling or heating at the working end of the semiconductor chilling plate can be achieved by the control of the control device 3; while the circulation passage 4 is formed in one side, away from the working surface of the compress bag 1, of the semiconductor chilling plate 2, and the circulation passage 4 exchanges heat with the semiconductor chilling plate 2 to achieve heat dissipation and the like, so as to ensure the temperature of the device to be stable.

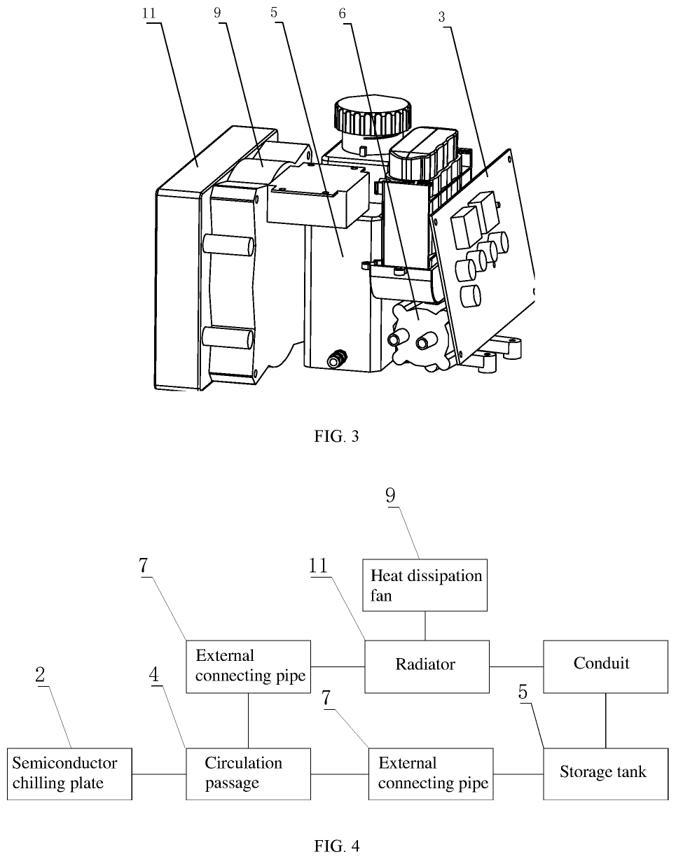

As shown in FIG. 2 and FIG. 3, in the arrangement of the cooling medium circulation device, the storage tank 5, the pumping device 6, and the external connecting pipe 7 are all externally disposed on the compress bag 1; wherein the cooling medium can be selected from the cooling water, a cooling gas and the like; the storage tank 5 is used for storing the cooling water, the external connecting pipe 7 is connected to the circulation passage 4 in the compress bag 1 and the storage tank 5, while the pumping device 6 is disposed on one side of the storage tank 5 and electrically connected to the control device 3.

The secondary heat dissipation device includes a heat dissipation fan 9 and a radiator 11, wherein the heat dissipation fan 9 is electrically connected to the control device 3, the heat dissipation fan 9 is used for blowing the radiator 11 for heat dissipation, and the radiator 11 is disposed on an air outlet path of the heat dissipation fan 9.

As shown in FIG. 1, the external connecting pipe 7 includes a water inlet pipe and a water outlet pipe, and specifically, one end of the water inlet pipe is connected to the storage tank 5, and the other end of the water inlet pipe is connected to a water inlet end of the circulation passage 4; and one end of the water outlet pipe is communicated with the radiator 11, the radiator 11 is connected to the storage tank 5, and the other end of the water outlet pipe is connected to a water outlet end of the circulation passage 4.

As shown in FIG. 4, after the cooling water which absorbs the working heat of the semiconductor cooling and heating plate is pumped out of the compress bag 1 by the pumping device 6, the cooling water flows into the water outlet pipe and continues to flow to the radiator 11 along the water outlet pipe, the radiator 11 exchanges heat with the cooling water, and the heat dissipation fan 9 blows the radiator 11 for heat dissipation, so as to ensure the cooling and subsequent recycling of the cooling water.

The control device 3, the storage tank 5, the pumping device 6, and the secondary heat dissipation device are disposed in the housing, and the components above are integrated through the housing, so as to facilitate replacement and management, wherein the control device 3 includes a display screen and a control button which are disposed on a surface of the housing, and a control circuit disposed in the housing, and the display screen and the control button are both electrically connected to the control circuit; and the circuit substrate 8 in the compress bag 1, an electric wire connected to the control circuit, the water outlet pipe, and the water inlet pipe are sleeved by a casing together, so as to avoid scattered distribution of conduits and lines.

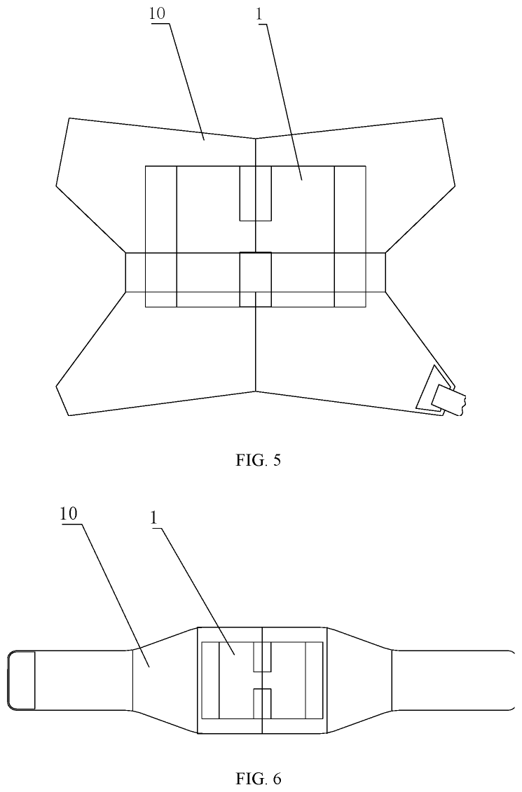

As shown in FIG. 5 to FIG. 7, the strap 10 acts as a fixing device when the compress bag 1 is in use and is removably connected to the compress bag 1 through a magic tape, and such design facilitates removal and adjustment of the position of the compress bag 1. The strap 10 can be selected from a suitable shoulder strap 10, waist strap 10, or limb strap 10 for a part requiring hot compress or cold compress.

A working principle of the present application is as follows: firstly, the required strap 10 is combined with the compress bag 1 through the magic tape, and then bound to a corresponding part of the human body, so that the working surface of the compress bag 1 is attached to the part of the human body; thereafter, according to the demand for cold compress and hot compress, selective adjustment is performed on the control device 3; during cold compress, the cooling of the working end of the semiconductor chilling plate 2 is controlled by the control device 3, the working end of the semiconductor chilling plate 2 directly exchanges heat with the part of the human body attached to the working surface of the compress bag 1, while heat dissipation and cooling of one side, away from the working surface of the compress bag 1, of the semiconductor chilling plate 2 is performed through the cooling medium circulation device. Specifically, the pumping device 6 is controlled by the control device 3 to pump the cooling water from the storage tank 5 to sequentially flow through the water inlet pipe, the water inlet end of the circulation passage 4 in the compress bag 1, the water outlet end of the circulation passage 4 in the compress bag 1, the water outlet pipe and the radiator 11, and finally flow back to the storage tank 5; the cooling water flowing in the circulation passage 4 can rapidly take away the working heat of the semiconductor chilling plate 2 due to a water route of the cooling water adjacent to the semiconductor chilling plate 2; and meanwhile, heat dissipation and cooling of the cooling water at the water outlet pipe is also performed by the control device 3 through the secondary heat dissipation device, so as to ensure the heat exchange efficiency of subsequent recycling.

In summary, for the device for cold compress and hot compress provided by the present application, through rapid cooling and heating of the semiconductor chilling plate, temperature adjustment on the working surface of the compress bag is achieved to meet the demand for cold compress and hot compress of a user; the semiconductor chilling plate is directly disposed on the working surface of the compress bag, so as to ensure that temperature adjustment on the semiconductor chilling plate can directly act on a desired part, and improve the heat exchange efficiency; and by disposing the cooling medium circulation device, working heat of the adjacent semiconductor chilling plate during cooling is taken away by flowing of the cooling medium in the circulation passage in the compress bag, so as to rapidly dissipate heat of the whole device to ensure that a working temperature is normal; while the cooling water can exchange heat with heat at a cold end on the other side of the semiconductor chilling plate during heating of the semiconductor chilling plate, so as to ensure temperature balance of the whole device, and prevent temperatures at two ends of the semiconductor chilling plate from affecting each other; the cooling medium can be cooling water, and the cooling water in a fluid form can satisfy efficient heat exchange in various models after the compress bag is attached to the human body; by adopting the matching of the cooling medium circulation device and the secondary heat dissipation device, heat exchange is rapidly performed on the semiconductor chilling plate, so as to ensure the temperature of the whole device to be stable; and by adopting the removable design of the strap and the compress bag, the operation and use of the user are further facilitated.

The description above is merely an example of the present application, and does not limit the patent scope of the present application accordingly. Any equivalent transformation made by using the contents of the description and the drawings of the present application, or direct or indirect application in the relevant technical field, is likewise included in the patent protection scope of the present application.

What is claimed is:

1. A device for cold compress and hot compress, comprising a compress bag, a control device, and a heat dissipation device, wherein
the compress bag comprises a semiconductor chilling plate and a working surface for being attached to a human body, and the semiconductor chilling plate is disposed on the working surface;
the heat dissipation device comprises a cooling medium circulation device, the cooling medium circulation device comprises a circulation passage and a cooling medium in the circulation passage, and the circulation passage is formed in the compress bag and is adjacent to one side, away from the working surface, of the semiconductor chilling plate; and
the control device is electrically connected to the semiconductor chilling plate and the heat dissipation device.

2. The device for cold compress and hot compress according to claim 1, wherein the compress bag further comprises a circuit substrate disposed corresponding to the semiconductor chilling plate, the semiconductor chilling plate is disposed on the circuit substrate, and the circuit substrate is electrically connected to the control device.

3. The device for cold compress and hot compress according to claim 1, wherein the cooling medium circulation device further comprises a storage tank, a pumping device, and an external connecting pipe, and the external connecting pipe is connected to the circulation passage in the compress bag and the storage tank; and the pumping device is disposed on one side of the storage tank and electrically connected to the control device.

4. The device for cold compress and hot compress according to claim 1, wherein the heat dissipation device further comprises a secondary heat dissipation device, and the cooling medium flows through the secondary heat dissipation device; and the secondary heat dissipation device is electrically connected to the control device.

5. The device for cold compress and hot compress according to claim 4, wherein the secondary heat dissipation device comprises a radiator and a heat dissipation fan, the cooling medium flows through the radiator, and the radiator is disposed on an air outlet path of the heat dissipation fan.

6. The device for cold compress and hot compress according to claim 1, further comprising a strap, wherein the strap is removably connected to the compress bag.
